(19) United States
(12) Patent Application Publication
(10) Pub. No.: US 2026/0365954 A1
(43) Pub. Date: Nov. 19, 2026

(54) DEVICES AND METHODS FOR THE QUANTIFICATION OF AGGREGATION

(71) Applicant: The Regents Of The University Of Michigan, Ann Arbor, MI (US)

(72) Inventors: Alexander RICKARD, Ann Arbor, MI (US); Michael HAYASHI, Ann Arbor, MI (US); Jason WING, Ann Arbor, MI (US); Kushbu Narender SINGH, Ann Arbor, MI (US)

(21) Appl. No.: 19/130,038

(22) PCT Filed: Nov. 15, 2023
(86) PCT No.: PCT/US2023/079868

§ 371 Date: May 14, 2025

Related U.S. Application Data
(60) Provisional application No. 63/383,872, filed on Nov. 15, 2022.
(60) Provisional application No. 63/384,038, filed on Nov. 16, 2022.

(51) Int. Cl. C12M 1/32; C12M 1/00; C12Q 1/06

(52) U.S. Cl. CPC C12M 23/12; C12M 23/22; C12Q 1/06

(57) ABSTRACT

Provided herein are devices comprising microwells (e.g., microplates) comprising a convex bottom geometry and methods of use thereof for the quantification of aggregation and the characteristics thereof. In particular, the devices herein find use in standard spectrophotometric plate readers and facilitate reproducible and high-throughput quantification of the strength and kinetics of microbial aggregation and coaggregation.

### CROSS-REFERENCE TO RELATED APPLICATIONS

The present invention claims priority to U.S. Provisional Patent Application Ser. No. 63/383,872, filed Nov. 15, 2022, and U.S. Provisional Patent Application Ser. No. 63/384,038, filed Nov. 16, 2022, both of which are incorporated by reference in their entireties.

### FIELD

Provided herein are devices comprising microwells (e.g., microplates) comprising a convex bottom geometry and methods of use thereof for the quantification of aggregation and the characteristics thereof. In particular, the devices herein find use in standard spectrophotometric plate readers and facilitate reproducible and high-throughput quantification of the strength and kinetics of microbial aggregation and coaggregation.

### BACKGROUND

In the field of microbiology, coaggregation refers to the recognition and adhesion of genetically distinct bacteria (Kolenbrander et al, 1993; Rickard et al, 2003a; incorporated by reference in their entireties). While originally detected between human oral bacteria (Gibbons & Nygaard, 1970; incorporated by reference in its entirety), it has now been shown to occur between microorganisms from a wide range of environments including the human urogenital tract, the skin of humans, and activated sludge (Datta et al, 2018; Kumar et al, 2019; Malik et al, 2003; Reid et al, 1988; incorporated by reference in their entireties). Coaggregation has been proposed to enhance freshwater biofilm development (Afonso et al, 2021; Rickard et al., 2003a; incorporated by reference in their entireties). Freshwater biofilms are of concern as they can aid in the retention and enrichment of pathogens (Hayward et al, 2022; Wang et al, 2021; incorporated by reference in their entireties), increase the bacterial load in the surrounding water (Douterelo et al, 2014; Liu et al, 2013; incorporated by reference in their entireties), promote microbial induced corrosion (Yang et al, 2012; incorporated by reference in its entirety), and reduce the chemical and cosmetic quality of water (Kerr et al, 2003; Zhou et al, 2017; incorporated by reference in their entireties). Interest in freshwater bacterial coaggregation began 25 years ago (Buswell et al, 1997; incorporated by reference in its entirety) and cumulative evidence from different research groups has indicated that coaggregation occurs between numerous freshwater bacterial species (Cheng et al, 2014; Rickard et al, 2002; Simoes et al, 2008; Vornhagen et al, 2013; incorporated by reference in their entireties) and is often mediated by highly specific complementary adhesin-receptor interactions (Rickard et al, 2000; Simoes et al., 2008; incorporated by reference in their entireties). In addition, two studies from two independent research groups have indicated that under in vitro conditions, coaggregation between freshwater bacteria enhanced biofilm development (Min & Rickard, 2009; Simoes et al., 2008; incorporated by reference in their entireties).

Bacterial coaggregation is typically measured using a tube-based visual assay (Cisar et al, 1979; incorporated by reference in its entirety). An ordinal scale of 0-4 is used to represent the strength of coaggregation in the assay (Cisar et al., 1979; Rickard et al., 2000; incorporated by reference in their entireties). While this method is accessible, the coaggregation score assigned is only semi-quantitative due to differences in subjective judgment between observers, which may impair rigorous comparisons between different strains or organisms. In addition, visual coaggregation assays are not high throughput and do not explicitly measure the time dynamics of coaggregation. Fully quantitative methods have been proposed using image analysis (Bos et al, 1994; Levin-Sparenberg et al, 2016; incorporated by reference in their entireties), cuvette spectrophotometry (Karched et al, 2015; Malik et al., 2003; incorporated by reference in their entireties), and microplate spectrophotometry (Metzger et al, 2001; incorporated by reference in its entirety). These methods vary in terms of cost, complexity, throughput, and capacity to measure coaggregation over time (Afonso et al., 2021; incorporated by reference in its entirety). For example, cuvette spectrophotometry is relatively inexpensive and simple to perform, but offers low throughput. Image analysis methods require highly specialized equipment and are computationally expensive. Finally, microplate spectrophotometry is susceptible to variation in experimental procedures that lead to statistically inconsistent results.

Coaggregation is an understudied phenomenon in freshwater biofilm development (Afonso et al., 2021; Katharios-Lanwermeyer et al, 2014; incorporated by reference in their entireties). Studies have been hampered in part due to methodological issues—for example the method to quantify coaggregation, or the use of a particular type of buffer to study coaggregating cells (Min & Rickard, 2009; incorporated by reference in its entirety)—but also due to a lack of appreciation for how coaggregation may be important in freshwater biofilm development and its potential use in biofilm control. For example, the possibility of using coaggregating probiotic urogenital lactobacilli to create a localized hostile environment around adjacent pathogens is being explored (Pashayan & Hovhannisyan, 2021; Reid et al., 1988; Younes et al, 2012; incorporated by reference in their entireties). With increasing concerns regarding the overuse of antibiotics in the freshwater environment (Hernandez et al, 2007; Monteiro & Santos, 2020; Osorio et al, 2016; incorporated by reference in their entireties) and the potential negative environmental impact of the overuse of biocides (Cloete & Flemming, 2012; Durak et al, 2021; Guardiola et al, 2012; Kahrilas et al, 2015; incorporated by reference in their entireties), novel strategies that can specifically target problematic freshwater biofilm species by targeting their coaggregation abilities may also offer exciting sustainable alternatives to current chemical control strategies (Afonso et al., 2021; incorporated by reference in its entirety).

When considering the need to further explore the fundamental importance of coaggregation and its potential use in freshwater biofilm control, a robust (e.g., replicable and reproducible) method to quantitatively measure coaggregation is needed.

### SUMMARY

Provided herein are devices comprising microwells (e.g., microplates) comprising a convex bottom geometry and methods of use thereof for the quantification of aggregation and the characteristics thereof. In particular, the devices herein find use in standard spectrophotometric plate readers and facilitate reproducible and high-throughput quantification of the strength and kinetics of microbial aggregation and coaggregation.

In some embodiments, provided herein are microwells comprising a convex interior bottom surface extending to the periphery of the microwell. In some embodiments, the microwell comprises one or more side walls enclosing the periphery of the microwell. In some embodiments, the microwell comprises a single wall enclosing the periphery of the microwell. In some embodiments, the microwell has a circular or oval cross-section. In some embodiments, the microwell comprises a multiple walls enclosing the periphery of the well. In some embodiments, the microwell has a polygonal cross-section (e.g., triangle, square, pentagon, hexagon, octagon, etc.). In some embodiments, the microwell comprises an open top.

In some embodiments, the entire interior bottom surface is convex and not comprising any planar portions. In some embodiments, the center of the cross-section of the bottom surface of the microwell is the highest point on the bottom surface. In some embodiments, the bottom surface extends continuously down from the center point to the periphery without local increases in height. In some embodiments, the convex interior bottom surface is parabolic (e.g., increasing rate of downward curvature extending outward from the center). In some embodiments, the convex interior bottom surface has constant curvature extending outward from the center (e.g., approximating a portion of a sphere).

In some embodiments, a microwell herein has a volume of 100 μl to 5 ml (e.g., 100 μl, 200 μl, 500 μl, 1 ml, 1.5, ml, 2 ml, 3 ml, 4 ml, 5 ml, or ranges therebetween).

In some embodiments, the convex interior bottom surface is transparent. In some embodiments, the microwell (e.g., bottom, sides, etc.) is transparent. In some embodiments, the microwell comprises polystyrene, polypropylene, polycarbonate, polyurethane, acrylonitrile butadiene styrene (ABS), glass, cyclo-olefins, or a combination thereof.

In some embodiments, provided herein are devices comprising one or more of the microwells described herein. In some embodiments, provided herein are microplates comprising one or more of the microwells described herein. In some embodiments, a microplate comprising a planar surface (e.g., top surface) displaying a plurality of the microwells herein. In some embodiments, a microplate comprises 2-1000 microwells (e.g., 2, 4, 6, 8, 12, 16, 24, 48, 96, 384, or more). In some embodiments, the microwells are cavities or depressions in the planar surface.

In some embodiments, a device or microplate comprises a lip or cover for the microwells. In some embodiments, the microplate comprises a single cover or lid that provides a top to all of the microwells on the microplate. In some embodiments, each microplate comprises a separate cover or lid. In some embodiments, the cover or lip is transparent.

In some embodiments, provided herein are methods of analyzing a sample comprising: adding the sample (or a portion thereof) to a microwell herein (e.g., a microwell of a microplate; and using a detection instrument to detect a physical or chemical characteristic of the sample in the microwell. In some embodiments, the instrument measures the physical or chemical characteristic of the sample from above or below the microwell or microwells. In some embodiments, the instrument measures and/or detects an optical characteristic of the sample. In some embodiments, the instrument is a spectrophotometer, colorimeter, camera, or other device capable of detecting and/or quantitating an optical characteristic of the sample within the microwell. In some embodiments, the instrument is a spectrophotometer and the physical or chemical characteristic is optical density.

In some embodiments, a sample is a biological sample, an environmental sample, an laboratory sample, etc. A sample, may be a freshwater environmental sample, such as water from a natural source (e.g., lake, river, etc.) or water from a manmade source, such as wastewater, municipal water, etc. In some embodiments, the aggregation (e.g., coaggregation) of components within a sample is monitored. In some embodiments, the aggregation of non-living components is monitored, such as particulate matter, precipitates, protein or lipid matter, etc. In some embodiments, the aggregation of living components is monitored, such as microbes, etc.

In some embodiments, the instrument measures the physical or chemical characteristic at the center of the microwell. In some embodiments, the physical characteristic is measured within the center 25% (e.g., 20%, 15%, 10%, 5%, 2%, 1%, etc.) of the microwell (e.g., % determined linearly from the center in any cross-sectional dimension). In some embodiments, the instrument does not measure the physical or chemical characteristic at the periphery of the microwell. In some embodiments, the instrument does not measure the physical or chemical characteristic within the outer 25% (e.g., 20%, 15%, 10%, 5%, 2%, 1%, etc.) of the microwell e.g., % determined linearly from the sidewall in any cross-sectional dimension).

In some embodiments, the sample comprises one or more components with a density greater than the liquid of the sample. In some embodiments, because of the convex shape of the interior bottom surface of the microwells herein, components with a density greater than the liquid of the sample fall to the bottom of the microwell and concentrate at the periphery of the microwell.

In some embodiments, the sample comprises one or more components capable of aggregation. In some embodiments, the sample comprises one or more components capable of autoaggregation. In some embodiments, the sample comprises one or more components capable of coaggregation. In some embodiments, aggregates of the one or more components fall away from the center of the microwells herein along the convex interior bottom surface toward the periphery of the microwell or microwells.

In some embodiments, a sample comprises one or more types of microbes (e.g., bacteria, archaea, fungi, protists, viruses, etc.). In some embodiments, the sample comprises one or more species of bacteria. In some embodiments, the sample comprises spherical (cocci), rod (bacilli), spiral (spirilla), comma (vibrios), and/or corkscrew (spirochaetes) shaped bacteria. In some embodiments, the bacteria are biofilm-forming bacteria (e.g., Enterococcus faecalis, Staphylococcus aureus, Staphylococcus epidermidis, Streptococcus viridans, E. coli, Klebsiella pneumoniae, Proteus mirabilis Pseudomonas aeruginosa, etc.). In some embodiments, the bacteria are freshwater bacteria (e.g., Proteobacteria, Cyanobacteria, Actinobacteria, and Bacteroidestes, etc.). Embodiments herein are not limited by the nature or types of bacteria present in the sample. In some embodiments, one or more bacteria present in the sample are capable of antiaggregating. In some embodiments, autoaggregated bacteria concentrate at the bottom of the microwells herein (i.e., the periphery of the microwell). In some embodiments, two or more bacteria present in the sample are capable of coaggregating. In some embodiments, coaggregated bacteria concentrate at the bottom of the microwells herein (i.e., the periphery of the microwell). In some embodiments, as bacteria within the sample autoaggregate or coaggregarte, the concentration of bacteria in the center of the microwell (cross-sectionally) is reduced as the aggregated bacteria fall to the bottom of the microwell, thereby concentrating at the periphery. An analytical (e.g., biophysical) technique that is capable of detecting (e.g., quantifying) the amount and/or concentration of bacteria, when performed at the center of a microwell herein, will detect decreasing amount or concentration of bacteria as the aggregated bacterial falls to the bottom/periphery of the microwell.

In some embodiments, methods herein further comprise allowing the sample to incubate within the microwell or microwells. In some embodiments, the sample is allowed to incubate under conditions that allow microbes (e.g., bacteria) to grow (e.g., culture conditions). In some embodiments, the sample is allowed to incubate under conditions that allow microbes (e.g., bacteria) to aggregate. In some embodiments, the detection and/or quantification of a chemical or physical characteristic of the sample (e.g., optical density at the cross-sectional center of the microwell) is performed at multiple (e.g., 2, 5, 10, 100, 200, 500, or more) timepoints (e.g., every second, 5 seconds, 10 seconds, 15 seconds, 30 seconds, minute, 2 minutes, 5 minutes, 10 minutes, or more, or ranges therebetween), for example, during or following the incubation.

In some embodiments, methods herein further comprise steps of analyzing the data collected during methods steps performed (e.g., optical density of sample) to determine the amount or concentration of one or more species of bacteria, total bacteria, unaggregated bacteria, etc. in the sample. In some embodiments, analysis methods further comprise calculating the kinetics of aggregation (e.g., autoaggregation of bacteria, coaggregation of bacteria, etc.) based on the change in the physical or chemical characteristic measured at the center of the microwell or microwells over time. In some embodiments, method comprise calculating the kinetics of coaggregation within the sample. In some embodiments, analysis method comprise calculating the rate of aggregation (e.g., autoaggregation of bacteria, coaggregation of bacteria, etc.) based on the change in the physical or chemical characteristic measured at the center of the microwell or microwells over time. In some embodiments, methods comprise calculating the rate of coaggregation.

### BRIEF DESCRIPTION OF THE DRAWINGS

FIG. 1A-B. A 24-well microplate (test system) that contains dome shaped wells (12 on the left side of the microplate) and flat wells (12 on the right side of the plate). A. Diagram of the microplate, shown in the traverse (X-Y) and longitudinal (X-Z) dimensions. B. Photographs of a microplate shown in the traverse and longitudinal dimensions. Red dotted line highlights the domed or flat surface shape in the wells.

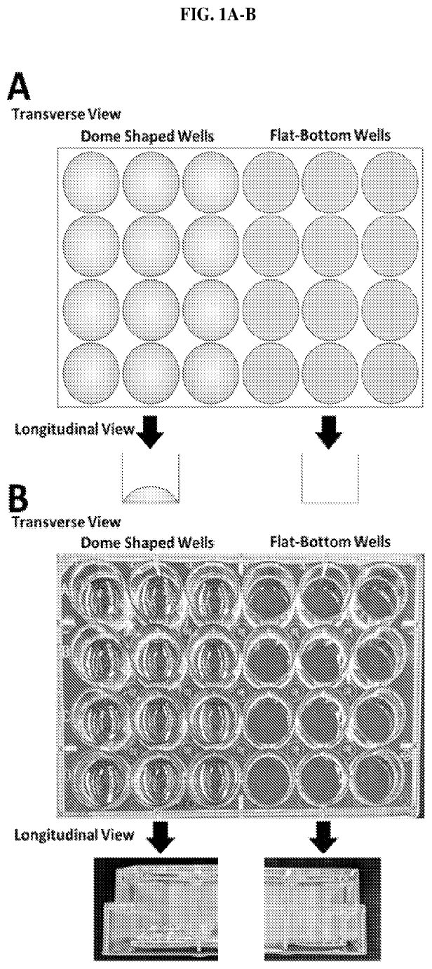

FIG. 2A-B. Visualization of coaggregation between B. natatoria 2.1 and M. luteus 2.13, that were grown at 30 °C or 37 °C, washed, and suspensions pipetted in to the microplate that contained dome shaped wells (DSWs) and flat-bottom wells. Single-(B. natatoria 2.1 or M. luteus 2.13) and dual-species (B. natatoria 2.1 and M. luteus 2.13) suspensions in the DSWs (left side 12 wells shown in A and B) and flat-bottom wells (right side wells shown in A and B). Photographs were taken at the end of microplate-based spectrophotometry assays.

FIG. 3A-F. Microscopic comparison, using confocal laser scanning microscopy, of the cellular arrangement of single-species cell suspensions and dual-species mixtures following growth as 30 °C or 37 °C. Green-fluorescent-colored cells are B. natatoria 2.1 (expressing GFP) and red-fluorescent-colored cells (stained with hexidium iodide) are M. luteus 2.13. Following growth at 30 °C, (A) strings of rosettes of B. natatoria 2.1 cells, (B) small clumps of tetrads of M. luteus 2.13 cells and, (C) large coaggregates of B. natatoria 2.1 and M. luteus 2.13. Following growth at 37 °C, (D) elongated rod-shaped cells of B. natatoria 2.1, (E) Large clumps of tetrads of M. luteus 2.13 cells and, (F) small coaggregates of B. natatorial 2.1 and M. luteus 2.13. Scale bar=20 um.

FIG. 4A-B. Percent differences between starting and ending optical density of single and dual-species suspensions within the DSWs and flat-bottom wells after the strains were grown at either 30 °C or 37 °C. Boxes represent 25th and 75th percentiles across experimental repetitions. Center lines represent median percent differences. (A) DSWs and (B) Flat-bottom wells. Data was from 5 experimental sets comprising a total of 20 experimental repetitions with each single or dual-species suspension being evaluated in 4 DSWs and 4 flat-bottom wells per experimental set. Median coaggregation strength in DSWs was approximately 67 at 30 °C and 16 at 37 °C (A). In flat-bottomed wells median coaggregation strength was approximately 53 at 30 C and 2 at 37 C (B). Visual coaggregation scores ranged between 3-4 at 30 °C and between 0-1 at 37 °C.

FIG. 5A-D. Optical density curves for dual-species coaggregating suspensions using DSWs and flat-bottomed wells in a spectrophotometric assay. Each data series represents a single experimental repetition (well). A. DSWs with organisms incubated at 30 °C, B. DSWs with organisms incubated at 37 °C, C. Flat-bottomed wells with organisms incubated at 30 °C, D. Flat-bottom wells with organisms incubated at 37 °C.

FIG. 6A-D. Best fit coaggregation kinetics models for 5 experimental sets. Kinetics parameters were estimated for each experimental set, using data from all repetitions in the set (20 total). A. DSWs with organisms incubated at 30 °C, B. DSWs with organisms incubated at 37 °C, C. Flat-bottomed wells with organisms incubated at 30 °C, D. Flat-bottom wells with organisms incubated at 37 °C.

FIG. 7. Examples of tubes containing single-species and dual-species suspensions that were assessed for autoaggregation (B. natatoria 2.1 or M. luteus 2.13) or coaggregation (B. natatoria 2.1 mixed with M. luteus 2.13). Cell suspensions were from cultures of B. natatoria 2.1 or M. luteus 2.13 grown in R2A at 30 °C or 37 °C, washed by centrifugation, and set to an optical density at 600 nm of 1.5.

FIG. 8. Illustration of coaggregation of bacteria in DSWs (left) vs. flat-bottom (right) microwells and exemplary kinetic data derived from each.

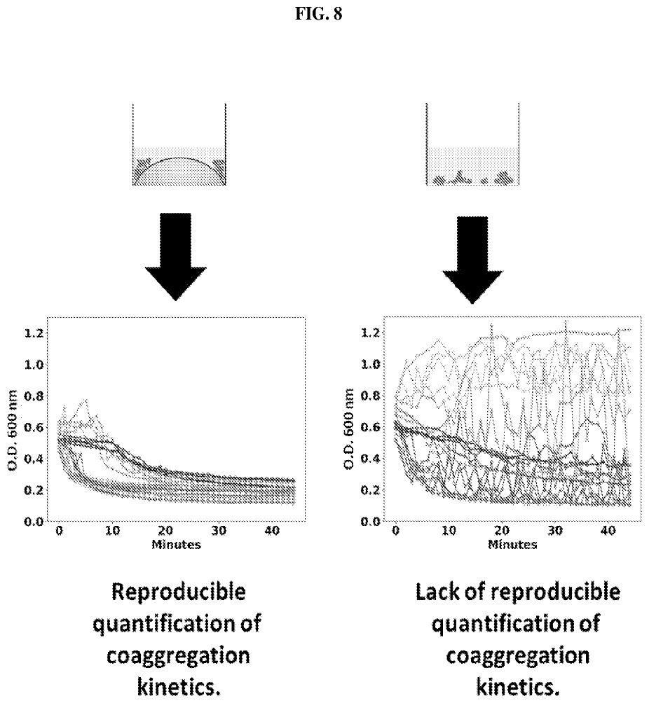

FIG. 9A-D. Microplate-based spectrophotometry assay of dental bacteria in buffered KCl or coaggregation buffer. Changes in optical density of mixtures of coaggregating oral bacteria Streptococcus gordonii DL1 and Actinomyces oris T14V in flat-bottomed wells (A,B) or dome shaped wells (C,D) over 25 time points in a microplate reader. Each data series represents a single experimental repetition (well). Two wells per plate per buffer had flat bottoms while two wells per plate per buffer had domed bottoms. 11 total experimental sets were included spanning 7 days. (A) and (C) represent experiments performed using suspensions in coaggregation buffer. (B) and (D) represent experiments using suspensions in buffered KCl. Black lines denote best fit coaggregation kinetics models. DSWs with buffered KCl yielded the overall best model fit (NLL=414.35).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a microwell” is a reference to one or more microwells and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “about,” when referring to a value is meant to encompass variations of ±10%, unless otherwise stated herein.

The terms “microwell” and “well” are used synonymously herein, and refer to a cavity on a surface capable of holding a volume of liquid. Microwells/wells may be made of various volumes, ranging from 100 μl to 5 ml.

As used herein, the terms “microplate”, “microtiter plate”, and “microplate” refer to any planar substrate ((“plate”) displaying multiple cavities (“microwells”) on a surface. The cavities are typically arranged in a regular array (e.g., rows and columns). A surface may display, for example, 6, 12, 24, 48, 96, 384, or any other suitable number of microwells. Samples to be analyzed are placed in the microwells and the entire plate and/or the individual microwells may be subjected to experimental conditions (e.g., for bacterial growth), chemical reaction, processing, and/or analysis (e.g., quantification).

As used herein, the term “convex” means outwardly bending or curving, such as the exterior surface of a sphere or parabola.

As used herein, the term “sample” is used in its broadest sense. In one sense, it is meant to include a specimen or culture obtained from any source as well as biological and environmental samples. Biological samples may be obtained from animals (including humans) and encompass fluids, solids, tissues, and gases. Biological samples include blood products such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the term “aggregation” refers to the clustering or clumping together of two or more entities. An aggregate may be formed of particulate matter within a sample, such as precipitates, flocculent masses, corpuscles, proteins, lipids, cellular debris, cells, microbes (e.g., bacteria, archaea, fungi, protists, viruses, etc.)

As used herein, the term “autoaggregation” refers to the clustering or clumping of two or more of the same entities together. For example, “bacterial autoaggregation” is the formation of aggregates from two or more of the same species (or strain) of bacteria.

As used herein, the term “coaggregation” refers to the clustering or clumping of two or more different entities together. For example, “bacterial coaggregation” is the formation of aggregates from two or more of different species (or strain) of bacteria.

As used herein, the term “microbe” refers broadly to any microscopic organisms, including bacteria, archaea, fungi, protists, and viruses.

### DETAILED DESCRIPTION

Provided herein are devices comprising microwells (e.g., microplates) comprising a convex bottom geometry and methods of use thereof for the quantification of aggregation and the characteristics thereof. In particular, the devices herein find use in standard spectrophotometric plate readers and facilitate reproducible and high-throughput quantification of the strength and kinetics of microbial aggregation and coaggregation.

Bacterial coaggregation, the adhesion of genetically distinct bacteria into a clump or cluster, is proposed to contribute to the development of freshwater biofilms. Freshwater biofilms can aid in the retention and survival of pathogens, reduce water quality, and can foul and damage surfaces. Methods to identify and quantify coaggregation typically rely on the semi-quantitative scoring of coaggregation in a tube-based visual aggregation assay. Provided herein are microplate-based systems and methods capable of, for example, measuring and modeling the kinetics of freshwater bacterial coaggregation. In experiments conducted during development of embodiments herein, the coaggregating freshwater bacterial strains Blastomonas natatoria 2.1 and Micrococcus luteus 2.13, that were originally isolated from an in vitro biofilm developed in water from a borehole water source, were evaluated for coaggregation ability using a tube-based visual aggregation assay and a 24-well microplate-based assay that contained convex bottom microwells (a.k.a., dome-shaped wells (DSWs)). Each 24-well microplate used in the exemplary experiments conducted during development of embodiments herein contained 12 flat-bottom wells and 12 DSWs to compare side-by-side and against the visual coaggregation scores in tubes. Confocal laser scanning microscopy was performed to visualize the size, structure, and arrangement of the cells within the coaggregates. In a spectrophotometric plate reader, the DSWs facilitated the reproducible detection of coaggregation between B. natatoria 2.1 and M. luteus 2.13. Quantitative coaggregation analysis using DSWs was also found to be more sensitive than the visual tube aggregation assay; barely detectable visual coaggregation scores were detected spectrophotometrically. Endpoint analyses of the data from flat-bottom wells and the DSWs demonstrated comparable mean coaggregation strengths and these correlated with the visual coaggregation scores. However, only the DSW could be used to estimate the kinetics of coaggregation. Furthermore, data collected from the flat-bottom wells exhibited high temporal and inter-replicate variance that yielded unreliable coaggregation kinetics estimates. Collectively these results demonstrate the utility of the DSW-based approach and improves upon the current toolkit for studying coaggregation between freshwater bacteria.

In some embodiments, provided herein are microwells having vertical sidewalls, an open top (or a closed or capped top), and a convex bottom surface. In certain embodiment, the microwells have a circular cross-sectional geometry and a single vertical sidewall surrounding the periphery of the microwell (e.g., extending vertically from the periphery of the bottom surface to the rim of the open top). The convex bottom surface comprises a single elevated point, typically at the center of the microwell. The bottom surface forms a regular curve from the elevated high point down to a low point at the periphery of the microwell, where the bottom surface meets the sidewall(s). The curve of the convex bottom may follow a single radius, may be parabolic, or may adopt another suitable curve. In some embodiments, the convex bottom contains no angles <180°, other than the angle between the bottom surface and the sidewall.

In some embodiments, a microwell has a circular cross-sectional geometry and a cross-sectional surface area of 0.25 to 10 cm² (e.g., 0.25 cm², 0.32 cm², 0.5 cm², 1.1 cm², 1.9 cm², 3.5 cm², 5 cm², 8 cm², 10 cm², or ranges therebetween). In some embodiments, a microwell has cross-sectional dimension(s) (e.g., width, diameter, etc.) of 3-30 mm (e.g., 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, or ranges therebetween). In some embodiments, a microwell has peripheral depth of (e.g., from top opening to bottom at periphery of the well) of 10-20 mm (e.g., 10 mm, 11 mm, 12 mm, 13 mm, 14 mm, 15 mm, 16 mm, 17 mm, 18 mm, 19 mm, 20 mm, or ranges therebetween). In some embodiments, the convex bottom surface of a microwell comprises a dome shape. In some embodiments the dome comprises a height (e.g., center peak height minus peripheral height) 2-8 mm (e.g., 2 mm, 3 mm, 4 mm, 5 mm, 6 mm, 7 mm, 8 mm, or ranges therebetween). In some embodiments, the dome height is 25% to 60% (e.g., 25%, 30%, 35%, 40%, 45%, 50%, 55%, 60%, or ranges therebetween) of the width of the microwell. In some embodiments, a microwell of a typical 24-well culture plate is about 14 mm wide and about 16 mm deep; an exemplary convex bottom surface for such a microwell comprises a dome with a width of 14 mm and a height of 4-6 mm (although other heights and widths are within the scope herein. As another example, a microwell of a typical 96-well microplate is about 5 mm wide and about 11 mm deep; an exemplary convex bottom surface for such a microwell comprises a dome with a width of 5 mm and a height of 2-4 mm (although other heights and widths are within the scope herein.

In some embodiments, the height of the dome of the convex bottom surface and the curvature of the dome to the periphery is sufficient to allow aggregated material to cascade down the dome, away from the center of the microwell, and collect at the periphery of the microwell.

In some embodiments, the convex bottom surface is an insert that can be placed in a flat-bottom microwell to form a convex-bottomed microwell. In some embodiments, the insert is sized to fit in the bottom of the microwell (e.g., bottom of insert is same size as the bottom of the microwell). In some embodiments, the insert is held to the bottom of the well with an adhesive. In some embodiments, the insert rests, unadhered, upon the bottom of the well. In some embodiments, any standard (or non-standard sized) flat-bottom microwell can find use in embodiments herein. Suitable microwells (e.g., within microplates or culture plates) are commercially available and understood in the field. In some embodiments, the widest diameter of the insert approximates (e.g., is equal to) the inner diameter of the bottom of the microwell. In some embodiments, the insert forms a seal with the sidewall(s), thereby not allowing fluid to travel from above the insert into the void created beneath the insert and above the flat bottom. In other embodiments, the convex bottom surface is part of the microwell and physically attached to the sidewall(s).

Dense components (e.g., greater density than water, the buffer, or the media present in the microwell) fall to the bottom of the microwell. In some embodiments, as the dense components contact the convex bottom surface, they continue to descend to the deepest point(s) of the microwell, at the periphery of the microwell. In some embodiments, the convex bottom surface comprises a material that prevents sticking of the dense components to the elevated portions of the convex bottom surface. The convex bottom surface may comprise a low friction material and/or be coated with a material or film to reduce friction between the dense components and the convex bottom surface. In some embodiments, due to the geometry of the microwell (e.g., the convex bottom surface), dense components concentrate at the periphery of the microwell.

In exemplary embodiments, the dense components of a sample are microbes (e.g., bacteria). When unaggregated the bacteria do not fall to the bottom of the microwell and remain distributed through the liquid of the sample within the microwell. However, aggregated bacteria (congregated or autoaggregated) fall to the bottom of the microwell and concentrate (e.g., >70%, >80%, >90%, >95%) at the periphery of the microwell. As a result of the distribution of unaggregated bacteria throughout the sample and the concentration of aggregated bacteria at the periphery, a biophysical or other analytic method capable of quantifying the amount/concentration of bacteria near the center of the microwell (e.g., at or near the central vertical axis of the microwell) will detect a decreasing amount/concentration of bacteria in the sample as the proportion of aggregated bacteria in the sample increases. By measuring the amount/concentration of bacteria near the center of the microwell over time, a rate of aggregation and/or the kinetics of aggregation of bacteria in the sample can be calculated. In some embodiments, the rate, kinetics, amount, or concentration of aggregated bacteria (congregated or autoaggregated) can be compared between sample conditions (e.g., in the presence of anti-biofilm test agents). In some embodiments, exemplary methods for analyzing the rate and kinetics of aggregation are provided in the Experimental section herein.

In some embodiments, methods and devices are provided for measuring one or more characteristics of a sample. In some embodiments, a sample comprises microbes (e.g., bacteria) is a liquid sample media (e.g., water, buffer, media, etc.). Any suitable liquid for providing a sample may find use in embodiments herein. In some embodiments described herein, a coaggregation buffer, such as the one in Cisar et al. (Infect Immun 24:742-752; incorporated by reference in its entirety) is utilized. In some embodiments, the coaggregation buffer does not contain sodium azide. In some embodiments, the coaggregation buffer comprises 0.001 M tris(hydroxymethyl)aminomethane adjusted to pH 8.0, CaCl₂ (1×10⁻⁴ M), MgCl₂ (1×10⁻⁴ M), NaN₃ (0.02%), and NaCl (0.15 M). In some embodiments, the coaggregation buffer comprises 0.001 M tris(hydroxymethyl)aminomethane adjusted to pH 8.0, CaCl₂) (1×10⁻⁴ M), MgCl₂ (1×10⁻⁴ M), and NaCl (0.15 M). In some embodiments, a sample comprises buffered KCl, such as the formulation of Lamont et al. (Microbiology 140:867-872; incorporated by reference in its entirety). In some embodiments, the buffered KCl comprises 5 mM KCl, 2 mM K₂HPO₄, 1 mM CaCl₂), pH 6.0. Other buffers, buffer components, pH, concentrations of components, etc. are within the scope herein and may find use in embodiments herein.

### EXPERIMENTAL

### Example 1

### Freshwater Bacteria

### Materials and Methods

### Freshwater Strains and Growth Conditions

Blastomonas natatoria 2·1 (also previously described as Sphingomonas natatoria 2.1) and Micrococcus luteus 2·13 were originally isolated from a biofilm that was developed on a glass coupon in a two-stage chemostat (Buswell et al., 1997; incorporated by reference in its entirety) that was inoculated with borehole water (Porton, Salisbury, UK). The two strains were grown on R2A agar at 30 °C or in R2A broth (Reasoner & Geldreich, 1985; incorporated by reference in its entirety) with shaking at 200 rpm at 30 °C. Each strain was stored at −80 °C in 25% v/v glycerol.

### Visual Tube Aggregation Assay

Blastomonas natatoria 2.1 and Micrococcus luteus 2.13 were grown in R2A broth for 48 hours at 30 °C and 37 °C with shaking at 200 rpm. Using a method similar to Rickard and colleagues (Rickard et al., 2002; incorporated by reference in its entirety), cultures were subsequently washed in sterilized tap water and set to an optical density (OD) at 600 nm of 1.5 and, to assess coaggregation, mixed in equal volumes (500 μL) in 10×75 mm borosilicate glass tubes (VWR, Radnor, PA, USA). The tubes were then agitated and rolled for 60 seconds to allow the cells to evenly mix. Coaggregation was scored at one and ten minutes using the criteria described by Cisar and coworkers (Cisar et al., 1979; incorporated by reference in its entirety). Coaggregation was scored on a scale of 0 to 4 where, O was assigned when no visible coaggregation was observed in the cell suspension, 1 was assigned when small uniform coaggregates in suspension 2 was given when large coaggregates were observed but the suspension remained turbid, 3 was designated for suspensions that contained large coaggregates which settled rapidly to leave some turbidity in the supernatant, and 4 was assigned when large coaggregates formed and settled immediately to leave a clear supernatant. To assess the extent of autoaggregation (self-aggregation), 1 mL of cell suspensions containing Blastomonas natatoria 2.1 or Micrococcus luteus 2.13 were scored using the same criteria as used for assessing coaggregation (Rickard et al, 2003b; incorporated by reference in its entirety). Coaggregation and autoaggregation assays were performed on the same cultures and these cultures were also used for the microplate assays.

### Microplate-Based Spectrophotometry Assay

B. natatoria 2.1 and M. luteus 2.13 were grown in R2A broth with shaking at 200 rpm at 30 °C or 37 °C and 48 hours. Cultures were subsequently washed in sterilized tap water and set to an OD of 1.5 at 600 nm according to the method described above for the visual coaggregation assay. The microplate-based coaggregation assays were performed using a 24-well flat-bottom microplate that was modified to contain domed wells (12 wells with domed wells and 12 unmodified and containing flat wells). The domed wells are referred to as DSWs and were constructed by sealing transparent glass cabochons (Craftdady, Shenzhen, Guangdong Province, China) that were between 14.5-15 mm in diameter dome-side-up inside flat-bottom wells using polyurethane varnish (Minwax, Cleveland, Ohio, USA). To assess autoaggregation, 1 mL single-species suspensions (i.e., suspensions of B. natatoria 2.1 and M. luteus 2.13) were added to four flat-bottom wells and four dome-bottom wells per plate. To assess coaggregation, 0.5 mL of each single-species suspension (i.e., suspensions of B. natatoria 2.1 or M. luteus 2.13) was combined in four flat-bottom wells and four dome-bottom wells per plate. The kinetics of coaggregation was monitored using a Wallac Victor 3 plate reader (Perkin Elmer, Waltham, MA, US) set to photometry mode. Specifically, once the wells in the microplate were filled with the cell-suspensions, the microplate was gently transferred to the device and the optical density at 600 nm was monitored every minute for 45 minutes. Data was saved as an Excel file (Microsoft, Redmond, WA, USA) for downstream analyses.

### Microscopic Visualization of Coaggregate Structure

To visualize B. natatoria 2.1, constitutive GFP-expressing B. natatoria 2.1 (B. natatoria 2.1GFP; Min and Rickard, 2009; incorporated by reference in its entirety) cells were washed in water and set to an O.D. of 1.5 at 600 nm. To visualize M. luteus 2.13, cells were set to an O.D. of 1.5 at 600 nm, stained with 2 μL/mL hexidium iodide (Invitrogen, Waltham, Massachusetts), washed in water three times, and set to an O.D. of 1.5 at 600 nm. Individual cell suspensions, and mixtures of each species were then added to glass tunes that were used for visual coaggregation assays. Imaging of the structure of the single-species cell arrangements or coaggregates formed in cell suspensions in the glass tubes were performed using a Leica SPE (Leica Microsystems, Buffalo Grove, IL, USA) confocal laser scanning microscope equipped with a 40× objective (air, 0.85 N.A.). Images were rendered using IMARIS Version 7.3.1 software (Bitplane, Zurich, Switzerland) by using the Easy3D visualization software component. Renderings were performed on an i7 (Intel, Santa Clara, CA, USA) Windows 10 (Microsoft, Redmond, WA, USA) PC equipped with Radeon RX 580 graphics card (American multinational semiconductor, Santa Clara, CA, USA).

### Quantification of Freshwater Bacterial Coaggregation

Data from the microplate experiments that were saved in Excel (Microsoft, Redmond, WA, USA) files were analyzed using Python 3.8 using the Numpy, Scipy, and Pandas (Harris et al, 2020; Mckinney, 2010; Python_Software_Foundation, 2022; Virtanen et al, 2020; incorporated by reference in their entireties).

### Coaggregation Strength Coefficient

Absorbance data from each 24-well microplate-based spectrophotometric assay (each plate containing 12 DSW and 12 flat-bottom wells) was used to estimate the strength of coaggregation between B. natatoria and M. luteus. Strength of coaggregation was calculated as the percentage difference between initial and final OD:

$$Coaggregation Strength Coefficient = \frac{\vert {OD}_{0} - {OD}_{F} \vert}{{OD}_{0}} \times 100$$

To smooth variation in final OD for a given well, ODF was calculated using the mean of the final three time points for that well. The coaggregation strength coefficient was calculated for each well. Coaggregation strength coefficients were averaged over all replicates, stratified by organism and incubation temperature. Data was stratified by experiment day. Each well assayed on a given day represents a single experimental repetition; all repetitions on a given day comprise an experimental set.

### Kinetics of Bacterial Coaggregation

Bacterial coaggregation between two species can be described by the following chemical reaction system

x[A]+y[B]→[C]

where [A] and [B] are the concentration of individual species, x and y are the number of species A and B respectively in the coaggregate product and [C] is the concentration of coaggregate.

This yields the following system of differential equations

$$\frac{d [ A ]}{d t} = \frac{d [ B ]}{d t} = - {{k [ A ]}^{x} [ B ]}^{y}$$

$$\frac{d [ C ]}{dt} = {{k [ A ]}^{x} [ B ]}^{y}$$

where k is the coaggregation rate constant. The experimental system of B. natatoria 2.1 and Micrococcus luteus 2.13 was modeled using the following second order system

[B]+[M]=[C]

where [B] and [M] are the concentrations of B. natatoria and M. luteus respectively and [C] is the concentration of coaggregate. The model also includes a two-stage distributed delay before species can coaggregate. This delay represents time needed for even mixing of the assay fluid in each well. Our full kinetics model is therefore

$$\frac{d [ {B}_{0} ]}{dt} = - {k}_{1} [ {B}_{0} ]$$

$$\frac{d [ {B}_{1} ]}{dt} = {k}_{1} [ {B}_{1} ] - {k}_{2} [ {B}_{1} ]$$

$$\frac{d [ {B}_{2} ]}{dt} = {k}_{2} [ {B}_{1} ] - {k}_{c} [ {B}_{2} ] [ M ]$$

$$\frac{d [ M ]}{dt} = - {k}_{c} [ {B}_{2} ] [ M ]$$

$$\frac{d [ C ]}{d t} = {k}_{c} [ {B}_{2} ] [ M ]$$

where k₁ and k₂, are the distributed delay rate constants and kc is the coaggregation rate constant.

### Estimation of the Coaggregation Rate Constant

The relationship between the optical density of a fluid and the concentration of species in that fluid is described by the Beer-Lambert law,

OD=c[X]

where OD is the optical density (absorbance) of the fluid, c is a constant of proportionality (incorporating both the absorptivity and optical path length), and [X] is the concentration of a given species in the fluid. In the context of bacterial coaggregation, we take [X] to be the sum of the concentration of both bacterial species in a domed microplate well. As coaggregation occurs, the concentration of each species decreases and the aggregate mass falls to the side of the domed well bottom, leading to a proportional decrease in the OD of that well.

The coaggregation kinetics model was fit to OD data from the spectrophotometric assay using a Poisson likelihood function. Models were fit to each experimental set, using data from all repetitions in the set. The following measurement equation was used to account for the scaling constant of proportionality and the baseline OD of empty wells:

[symbol]

=c₁([B]+[M])+c₂

where [B] is the concentration of B. natatoria 2.1, [M] is the concentration of M. luteus 2.13, c₁ is the constant of proportionality, and c₂ is the baseline offset.

### Quantitative Validation of DSW Versus Visual Coaggregation Scores

Median coaggregation strength as measured by OD percent differences was compared to visual coaggregation scores assessed for each set's inoculum. Correlation coefficients were calculated to assess the degree of agreement between the microplate-based spectrophotometry assay and the visual coaggregation assay.

### Comparison Between DSW and Flat-Bottom Wells

Best-fit kinetics coefficients and negative log likelihoods were estimated and compared between paired DSW and flat-bottom well assays for each experimental set.

### Quantification of Dental Bacterial Coaggregation

The methodology above was also applied to exemplary experiments to quantify coaggregation of dental bacteria S. gordonii DL1 and A. oris T14V.

### Results

Macroscopic and Microscopic Examination of Single and Dual-Species Cell Suspensions After growth at 30 °C or 37 °C, and following washing in sterilized tap water, autoaggregation (self-aggregation) between B. natatoria 2.1 cells, between M. luteus 2.13 cells, and coaggregation between B. natatoria 2.1 and M. luteus cells was assessed by eye (macroscopically) using the tube-based aggregation assay and by eye in the microplate system. The cell shapes and arrangement of the cells was also studied by placing suspensions on microscope slides, covering with a coverslip, and image using a confocal laser scanning microscope (CLSM).

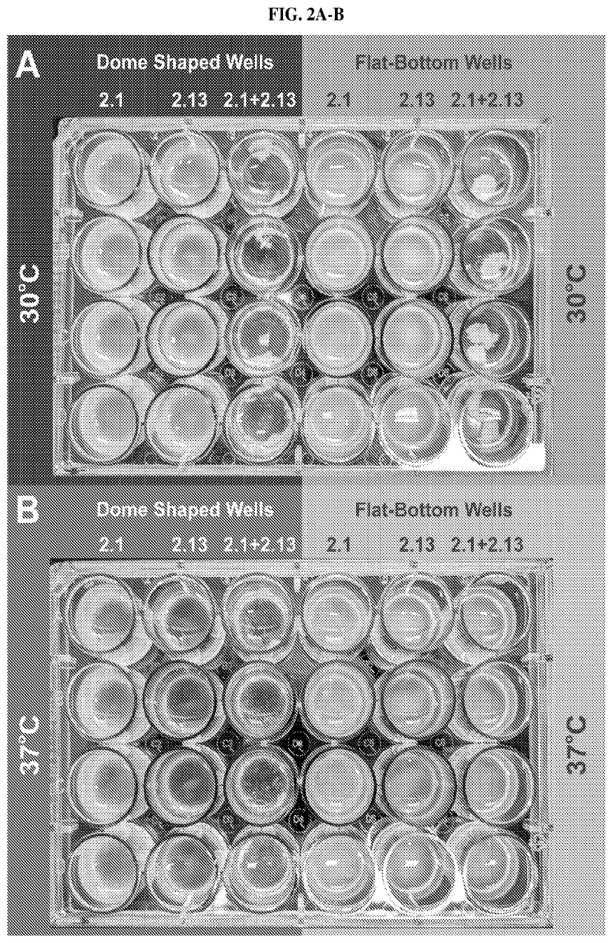

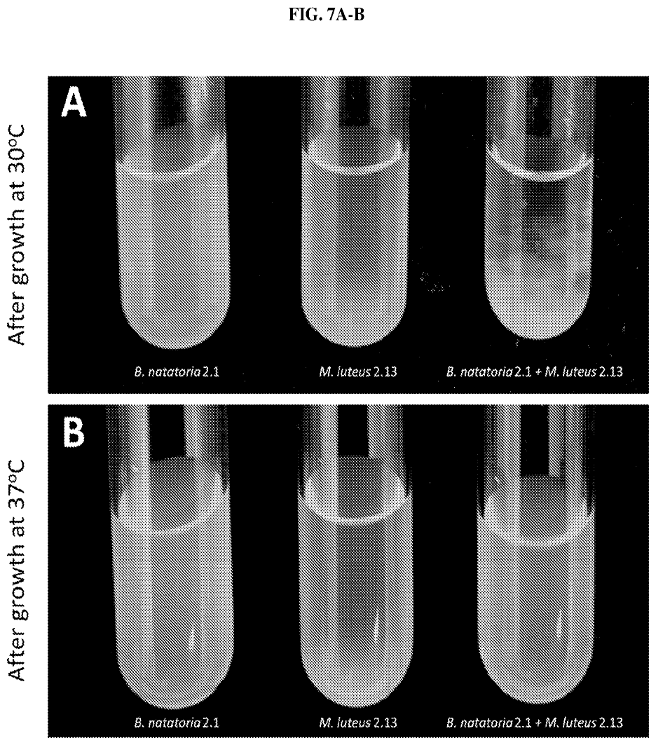

Coaggregation was observed by eye using the visual tube aggregation assay and the visual scores were assigned. Specifically, using the tube-based aggregation assay, after growth at 30 °C coaggregation occurred strongly between B. natatoria 2.1 and M. luteus 2.13 to yield an average score of 4. Large (>1 mm) aggregates formed, beginning to settle within 10 minutes and leaving a clear supernatant (FIG. 7A). In comparison, cells grown at 37 °C coaggregated poorly yielding a visual score varying between 0 or 1 depending on the experiment (FIG. 7B). No autoaggregation was observed by eye. Within DSW and flat microplate wells, a similar phenomenon was observed with clearly visible coaggregates forming in the two-organism mixture that had been grown at 30 °C as opposed to difficult to observe aggregates being seen between the B. natatoria 2.1-M. luteus 2.13 mixture that had been grown at 37 °C. Of particular note, the aggregates in the DSWs settled to the sides while the aggregates in the flat-bottom plates randomly settled on the flat surface (FIG. 2).

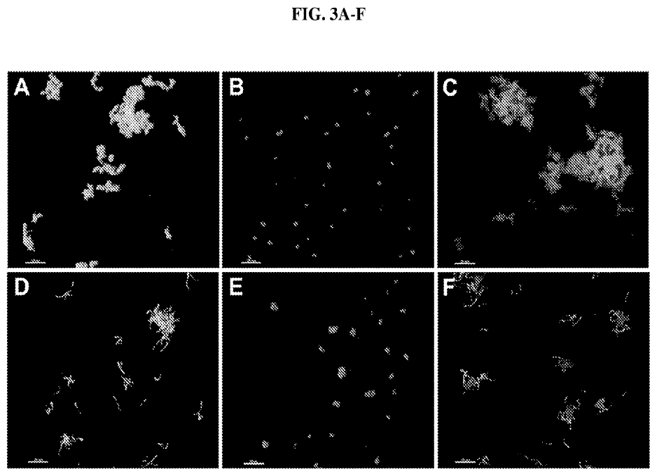

Cell-shapes, cell arrangements, and coaggregation ability were dependent upon the temperature at which they were grown. Specifically, when B. natatoria 2.1 and M. luteus 2.13 were grown at 30 °C, B. natatoria 2.1 cells grew as short rods and formed chains of rosettes (FIG. 3A) and M. luteus 2.13 formed loose or occasional small clumps of tetrads of cocci shaped cells (FIG. 3B). When mixed, the cells coaggregated to form large and often densely packed clumps (FIG. 3C). Conversely, when B. natatoria 2.1 was grown at 37 °C, cells grew as elongated rods that formed spider-like arrangements but, unlike cells grown at 30 °C, did not form chains of rosettes (FIG. 3D). After growth at 37 °C, M. luteus 2.13 formed tetrads of cocci, like cells grown at 30 °C, but clumps of tetrads tended to be larger (FIG. 3E). Coaggregation between B. natatoria 2.1 cells and M. luteus 2.13, grown at 37 °C, was substantially reduced compared to the cells grown at 30 °C prior to assessing for coaggregation (FIG. 3F).

### Quantification of Aggregation

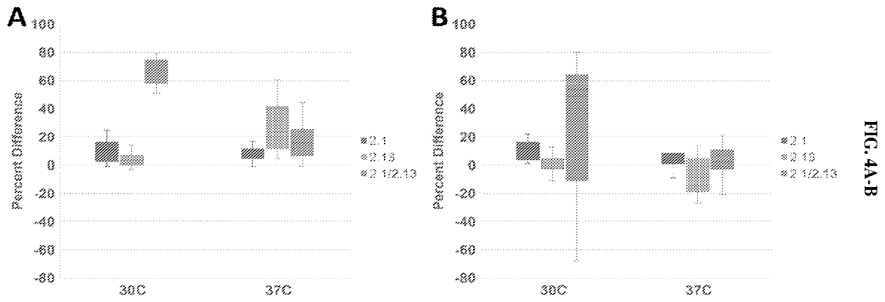

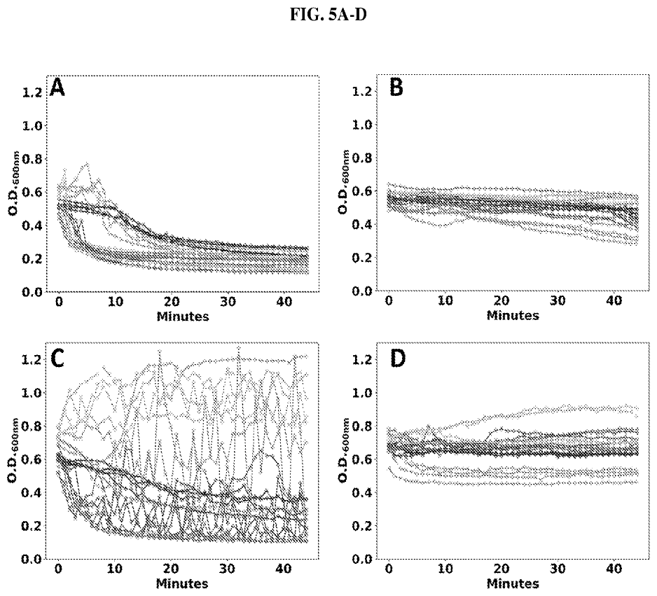

Autoaggregation (self-aggregation) of B. natatoria 2.1 cells and by M. luteus 2.13 cells, as well as coaggregation between B. natatoria 2.1 and M. luteus 2.13 cells was quantified using a microplate-based spectrophotometric assay for cells grown at 30 °C or 37 °C. The distribution of aggregation and coaggregation strengths, as inferred by changes in optical density, is shown in FIG. 4. Cells grown at 30 °C exhibited higher coaggregation strengths than those grown at 37 °C (median percent difference 67.45 vs 15.85). M. luteus 2.13 exhibited a stronger degree of autoaggregation when grown at 37 °C than 30 °C (median percent difference 23.47 vs 3.4). In the DSWs, coaggregation strengths were strongly correlated with visual coaggregation assay scores (Pearson correlation coefficient r=0.88 comparing set-average percent differences to paired set visual coaggregation assays). Compared to the DSWs, standard flat-bottom wells demonstrated a relatively similar median coaggregation strength for cells grown at 30 °C (53.44), but a substantial higher variance (25th-75th percentile interval=(−11.07, 63.61) for the flat-bottomed well vs (57.55, 74.21) for the DSW), notably ranging from strongly negative (−68.28) to strongly positive (79.25) percent differences. The coaggregation strengths in the flat-bottom wells were only moderately correlated with visual coaggregation scores (r=0.67). Both the DSWs and flat bottom wells yielded coaggregation scores that were significantly greater than zero (α=0.05), however, substantially higher overall variance from the flat-bottom wells suggests that this comparison is less robust than for DSWs. Spectrophotometry time series data is shown in FIG. 5. Data from the DSWs demonstrated consistent decreasing trends for cells grown at 30 °C while data from the flat-bottomed well plates was neither consistent within nor between repetitions.

### Model-Based Estimation of Coaggregation Rate

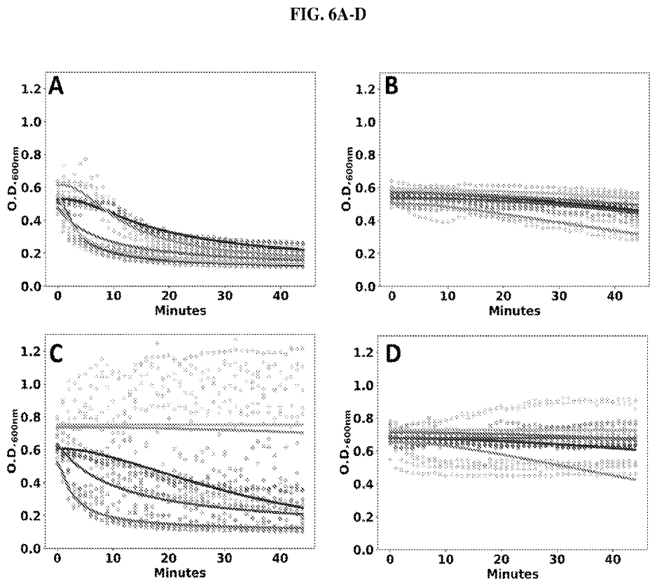

The coaggregation kinetics model was fit to time series data from the DSW spectrophotometric assays as well as time series data from flat-bottom well spectrophotometric assays. Models fit to DSW data yielded consistently good fits (mean Poisson negative log likelihood=105.18). The median rate constant estimated from DSW experimental sets for cells grown at 30 °C was 0.29 min⁻¹ (mean=0.39, range=0.137-−0.709). By contrast, models fit to data from flat bottom plates did not fit consistently well (mean Poisson negative log likelihood=139.87). This was reflected in the best fit values for the kinetics rate constant (median=1.19, mean=12456.63, range=0.179-43000.469). In the case of flat-bottom well model estimates, excessively high coaggregation rate constants indicate a practical estimation failure, even for reasonable NLL values, as near-instantaneous coaggregation is mechanistically implausible. Best fit model dynamics are shown in FIG. 6.

### Example 2

### Dental Bacteria

Further experiments were conducted on a pair of dental bacteria to assess their coaggregation and validate the DSW system. Data collection, processing, and analysis were conducted similarly to Example 1 with specific methods for the dental bacteria described below.

### Dental Strains and Growth Conditions

Streptococcus gordonii DL1 and A. oris T14V were grown overnight in Schaedler broth at 37 °C in 5% CO2. After washing by centrifugation three times in buffered KCl or coaggregation buffer, suspensions of S. gordonii DL1 or A. oris T14V were suspended in coaggregation buffer or buffered KCl at an optical density at 600 nm of 1.5 for subsequent aggregation assays.

### Microplate-Based Spectrophotometry Assay

A microplate coaggregation assay was performed using a Wallac Victor 3 microplate reader (Perkin Elmer). A 24-well microplate was used where 12 of the wells were unmodified flat-bottom wells and the other 12 wells were modified to be DSWs. The DSWs were constructed with glass cabochons that were 14.5 mm to 15 mm in diameter and fixed dome-side-up within the flat-bottom wells using polyurethane varnish. (Hayashi et al., 2023). For each experiment performed, single-species suspensions of S. gordonii DL1 or A. oris T14V, in coaggregation buffer or buffered KCl, were added to two DSWs and two flat-bottom wells using 1 mL of the respective suspension. On the same plate for each experiment, within two DSWs and two flat-bottom wells, suspensions of 0.5 mL of S. gordonii DL1 suspension and 0.5 mL of A. oris T14V suspension mixed for each buffer type to assess coaggregation. Upon adding the suspensions, the microplates were then placed into a microplate reader. There were no programmed shaking steps during the microplate readings and optical density at 600 nm was read 25 times with a delay of 2 minutes between each reading (i.e., 25 timepoints). Coaggregation kinetics were estimated for S. gordonii DL1 and A. oris T14V suspended in either coaggregation buffer or buffered KCl.

### Validation of DSW Spectrophotometry Assay for Dental Bacteria

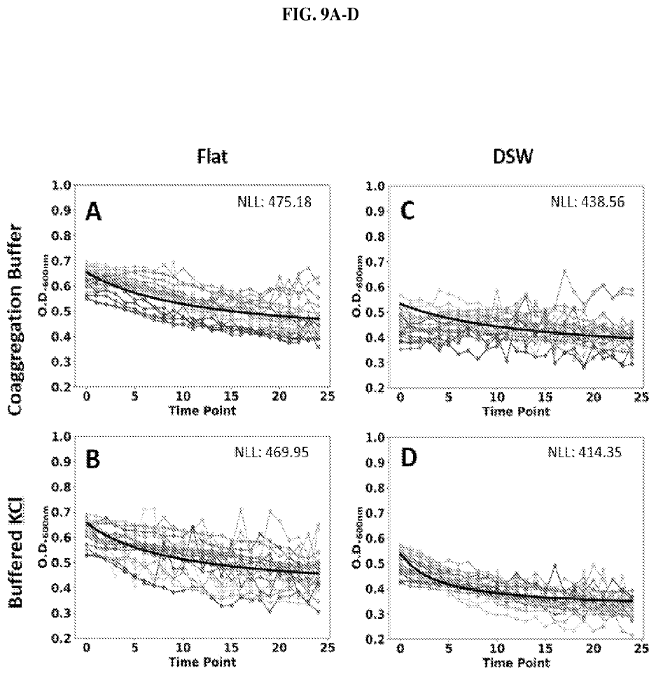

Model fits to data from DSWs and flat-bottom wells confirmed the ability of the DSWs to be used to consistently detect and quantify coaggregation in DSWs, with improved accuracy for suspensions in buffered KCl (FIG. 9).

### REFERENCES

The following references, some of which are cited above by author name, are herein incorporated by reference in their entireties.
Afonso A C, Gomes I B, Saavedra M J, Giaouris E, Simoes L C, Simoes M (2021) Bacterial coaggregation in aquatic systems. Water Res 196:117037
Bachrach G, Ianculovici C, Naor R, Weiss E I (2005) Fluorescence based measurements of Fusobacterium nucleatum coaggregation and of fusobacterial attachment to mammalian cells. Fems Microbiol Lett 248:235-240
Bos R, Vandermei H C, Meinders J M, Busscher H J (1994) A Quantitative Method to Study Co-Adhesion of Microorganisms in a Parallel-Plate Flow Chamber-Basic Principles of the Analysis. J Microbiol Meth 20:289-305
Buswell C M, Herlihy Y M, Marsh P D, Keevil C W, Leach S A (1997) Coaggregation amongst aquatic biofilm bacteria. J Appl Microbiol 83:477-484
Cheng Z, Meng X, Wang H, Chen M, Li M (2014) Isolation and characterization of broad spectrum coaggregating bacteria from different water systems for potential use in bioaugmentation. PLOS One 9: e94220
Cisar J O, Kolenbrander P E, McIntire F C (1979) Specificity of coaggregation reactions between human oral streptococci and strains of Actinomyces viscosus or Actinomyces naeslundii. Infect Immun 24:742-752
Cloete E, Flemming H (2012) Environmental Impact of Cooling Water Treatment for Biofouling and Biocorrosion Control. In: Operational and Environmental Consequences of Large Industrial Cooling Water Systems, Rajagopal S., Jenner H., Venugopalan V. (eds.) Springer, Boston, M A, USA:
Couvigny B, Kulakauskas S, Pons N, Quinquis B, Abraham A L, Meylheuc T, Delorme C, Renault P, Briandet R, Lapaque N et al (2018) Identification of New Factors Modulating Adhesion Abilities of the Pioneer Commensal Bacterium Streptococcus salivarius. Front Microbiol 9
Datta A, Stapleton F, Willcox M D P (2018) Bacterial Coaggregation and Cohesion Among Isolates From Contact Lens Cases. Invest Ophthalmol Vis Sci 59:2729-2735
Douterelo I, Husband S, Boxall J B (2014) The bacteriological composition of biomass recovered by flushing an operational drinking water distribution system. Water Res 54:100-114
Durak J, Rokoszak T, Skiba A, Furman P, Styszko K (2021) Environmental risk assessment of priority biocidal substances on Polish surface water sample. Environ Sci Pollut R 28:1254-1266
Feng D, Neuweiler I, Nogueira R, Nackenhorst U (2021) Modeling of Symbiotic Bacterial Biofilm Growth with an Example of the Streptococcus-Veillonella sp. System. Bull Math Biol 83:48
Gibbons R J, Nygaard M (1970) Interbacterial aggregation of plaque bacteria. Arch Oral Biol 15:1397-1400
Goeres D M (2010) Understanding the importance of biofilm growth in hot tubs. Applied Biomedical Microbiology: A Biofilms Approach: 133-148
Guardiola F A, Cuesta A, Meseguer J, Esteban M A (2012) Risks of Using Antifouling Biocides in Aquaculture. Int J Mol Sci 13:1541-1560
Harris C R, Millman K J, van der Walt S J, Gommers R, Virtanen P, Cournapeau D, Wieser E, Taylor J, Berg S, Smith N J et al (2020) Array programming with NumPy. Nature 585:357-362
Hayward C, Brown M H, Whiley H (2022) Hospital water as the source of healthcare-associated infection and antimicrobial-resistant organisms. Curr Opin Infect Dis 35:339-345
Hernandez F, Sancho J V, Ibanez M, Guerrero C (2007) Antibiotic residue determination in environmental waters by L C-M S. Trac-Trend Anal Chem 26:466-485
Hill K E, Malic S, McKee R, Rennison T, Harding K G, Williams D W, Thomas D W (2010) An in vitro model of chronic wound biofilms to test wound dressings and assess antimicrobial susceptibilities. J Antimicrob Chemoth 65:1195-1206
Kahrilas G A, Blotevogel J, Stewart P S, Borch T (2015) Biocides in Hydraulic Fracturing Fluids: A Critical Review of Their Usage, Mobility, Degradation, and Toxicity. Environ Sci Technol 49:16-32
Karched M, Bhardwaj R G, Asikainen S E (2015) Coaggregation and biofilm growth of Granulicatella spp. with Fusobacterium nucleatum and Aggregatibacter actinomycetemcomitans. BMC Microbiol 15:114
Katharios-Lanwermeyer S, Xi C, Jakubovics N S, Rickard A H (2014) Mini-review: Microbial coaggregation: ubiquity and implications for biofilm development. Biofouling 30:1235-1251
Kerr C, Osborn K, Rickard A, Robson G, Handley P (2003) Biofilms in water distribution systems. In: Water and wastewater engineering D M., N H. (eds.) pp. 757-776 . . . . Academic Press, London, United Kingdom:
Kocur M, Klooos W E, Schleifer K H (2006) The genus Micrococcus. In: The Prokaryotes, Dworkin M., Falkow S., Rosenberg E., Schleifer K. H., Stackebrandt E. (eds.) pp. 961-971.
Kolenbrander P E (1982) Isolation and Characterization of Coaggregation-Defective Mutants of Actinomyces-Viscosus, Actinomyces-Naeslundii, and Streptococcus-Sanguis. Infection and Immunity 37:1200-1208
Kolenbrander P E, Andersen R N, Holdeman L V (1985) Coaggregation of oral Bacteroides species with other bacteria: central role in coaggregation bridges and competitions. Infection and immunity 48:741-746
Kolenbrander P E, Ganeshkumar N, Cassels F J, Hughes C V (1993) Coaggregation: specific adherence among human oral plaque bacteria. FASEB J 7:406-413
Kumar K V, Pal A, Bai P, Kour A, E S, P R, Kausar A, Chatterjee M, Prasad G, Balayan S et al (2019) Co-aggregation of bacterial flora isolated from the human skin surface. Microb Pathog 135:103630
Ledder R G, Timperley A S, Friswell M K, Macfarlane S, McBain A J (2008) Coaggregation between and among human intestinal and oral bacteria. Fems Microbiology Ecology 66:630-636
Levin-Sparenberg E, Shin J M, Hastings E M, Freeland M, Segaloff H, Rickard A H, Foxman B (2016) High-throughput quantitative method for assessing coaggregation among oral bacterial species. Lett Appl Microbiol 63:274-281
Liu G, Verberk J Q, Van Dijk J C (2013) Bacteriology of drinking water distribution systems: an integral and multidimensional review. Appl Microbiol Biotechnol 97:9265-9276
Malik A, Sakamoto M, Hanazaki S, Osawa M, Suzuki T, Tochigi M, Kakii K (2003) Coaggregation among nonflocculating bacteria isolated from activated sludge. Appl Environ Microbiol 69:6056-6063
Mckinney W (2010) Data structures for statistical computing in python. Proceedings of the 9th Python in Science Conference 445:51-56
Melo L F, Pinheiro M M (1992) Biofouling in Heat-Exchangers. Nato Adv Sci Inst Se 223:499-509
Metzger Z, Featherstone L G, Ambrose W W, Trope M, Arnold R R (2001) Kinetics of coaggregation of Porphyromonas gingivalis with Fusobacterium nucleatum using an automated microtiter plate assay. Oral Microbiol Immun 16:163-169
Min K R, Rickard A H (2009) Coaggregation by the freshwater bacterium Sphingomonas natatoria alters dual-species biofilm formation. Appl Environ Microbiol 75:3987-3997
Min K R, Zimmer M N, Rickard A H (2010) Physicochemical parameters influencing coaggregation between the freshwater bacteria Sphingomonas natatoria 2.1 and Micrococcus luteus 2.13. Biofouling 26:931-940
Monteiro S, Santos R (2020) Incidence of enterococci resistant to clinically relevant antibiotics in environmental waters and in reclaimed waters used for irrigation. J Water Health 18:911-924
Osorio V, Larranaga A, Acena J, Perez S, Barcelo D (2016) Concentration and risk of pharmaceuticals in freshwater systems are related to the population density and the livestock units in Iberian Rivers. Sci Total Environ 540:267-277
Pashayan M M, Hovhannisyan H G (2021) Development of bifunctional vaginal suppositories by joint use terconazole and probiotic for treatment and prophylaxis of vulvovaginal candidiasis. Drug Dev Ind Pharm 47:1424-1431
Python_Software_Foundation, 2022. Python Language Reference, version 3.8.
Reasoner D J, Geldreich E E (1985) A New Medium for the Enumeration and Subculture of Bacteria from Potable Water. Appl Environ Microb 49:1-7
Reid G, McGroarty J A, Angotti R, Cook R L (1988) Lactobacillus inhibitor production against Escherichia coli and coaggregation ability with uropathogens. Can J Microbiol 34:344-351
Rickard A H, Gilbert P, Handley P S (2004) Influence of growth environment on coaggregation between freshwater biofilm bacteria. J Appl Microbiol 96:1367-1373
Rickard A H, Gilbert P, High N J, Kolenbrander P E, Handley P S (2003a) Bacterial coaggregation: an integral process in the development of multi-species biofilms. Trends Microbiol 11:94-100
Rickard A H, Leach S A, Buswell C M, High N J, Handley P S (2000) Coaggregation between aquatic bacteria is mediated by specific-growth-phase-dependent lectin-saccharide interactions. Appl Environ Microbiol 66:431-434
Rickard A H, Leach S A, Hall L S, Buswell C M, High N J, Handley P S (2002) Phylogenetic relationships and coaggregation ability of freshwater biofilm bacteria. Appl Environ Microbiol 68:3644-3650
Rickard A H, McBain A J, Ledder R G, Handley P S, Gilbert P (2003b) Coaggregation between freshwater bacteria within biofilm and planktonic communities. Fems Microbiol Lett 220:133-140
Simoes L C, Simoes M, Vieira M J (2008) Intergeneric coaggregation among drinking water bacteria: evidence of a role for Acinetobacter calcoaceticus as a bridging bacterium. Appl Environ Microbiol 74:1259-1263
Sly L I, Cahill M M (1997) Transfer of Blastobacter natatorius (Sly 1985) to the genus Blastomonas gen. nov. as Blastomonas natatoria comb. nov. Int J Syst Bacteriol 47:566-568
Sly L I, Hargreaves M H (1984) Two unusual budding bacteria isolated from a swimming pool. J Appl Bacteriol 56:479-486
Stevens M R, Luo T L, Vornhagen J, Jakubovics N S, Gilsdorf J R, Marrs C F, Moretro T, Rickard A H (2015) Coaggregation occurs between microorganisms isolated from different environments. FEMS Microbiol Ecol 91
Thomas T, Kindinger I, Yu D, Esvaran M, Blackall L, Forehead H, Johnson C R, Manefield M (2011) An experimental model for the spatial structuring and selection of bacterial communities. J Microbiol Meth 87:165-168
Virtanen P, Gommers R, Oliphant T E, Haberland M, Reddy T, Cournapeau D, Burovski E, Peterson P, Weckesser W, Bright J et al (2020) SciPy 1.0: fundamental algorithms for scientific computing in Python. Nat Methods 17:261-272
Vornhagen J, Stevens M, McCormick D W, Dowd S E, Eisenberg J N, Boles B R, Rickard A H (2013) Coaggregation occurs amongst bacteria within and between biofilms in domestic showerheads. Biofouling 29:53-68
Wang H, Hu C, Shi B (2021) The control of red water occurrence and opportunistic pathogens risks in drinking water distribution systems: A review. J Environ Sci (China) 110:92-98
Wang Q, Zhang T (2010) Review of mathematical models for biofilms. Solid State Communications 150:1009-1022
Wimpenny J, Colasanti R (2004) A simple cellular automaton model for coaggregation. Biofilms 1:369-375
Yang F, Shi B, Gu J, Wang D, Yang M (2012) Morphological and physicochemical characteristics of iron corrosion scales formed under different water source histories in a drinking water distribution system. Water Res 46:5423-5433
Younes J A, van der Mei H C, van den Heuvel E, Busscher H J, Reid G (2012) Adhesion Forces and Coaggregation between Vaginal Staphylococci and Lactobacilli. Plos One 7
Zhou X, Zhang K, Zhang T, Li C, Mao X (2017) An ignored and potential source of taste and odor (T&O) issues-biofilms in drinking water distribution system (DWDS). Appl Microbiol Biotechnol 101:3537-3550

1. A microwell comprising a convex interior bottom surface extending to the periphery of the microwell.

2. The microwell of claim 1, wherein the microwell comprises one or more side walls enclosing the periphery of the well.

3. The microwell of claim 2, wherein the microwell comprises a single wall enclosing the periphery of the well.

4. The microwell of claim 3, wherein the well has a circular cross-section.

5. The microwell of claim 1, wherein the microwell comprises an open top.

6. The microwell of claim 1, wherein the entire interior bottom surface is convex and not comprising any planar portions.

7. The microwell of claim 7, wherein the microwell has a volume of less than 1 ml.

8. The microwell of claim 7, wherein the microwell has a volume of less than 500 μL.

9. The microwell of claim 8, wherein the microwell has a volume of less than 50 μL.

10. The microwell of claim 1, wherein the convex interior bottom surface is transparent.

11. The microwell of claim 1, wherein the microwell is transparent.

12. The microwell of claim 1, wherein the microwell comprises polystyrene, polypropylene, polycarbonate, polyurethane, acrylonitrile butadiene styrene (ABS), glass, or cyclo-olefins.

13. A microplate comprising a planar surface displaying a plurality of the microwells, of one of claims 1-12.

14. The microplate of claim 13, wherein the planar surface comprises 4-16 microwells.

15. The microplate of claim 14, wherein the planar surface comprises 96 or fewer microwells.

16. The microplate of claim 15, wherein the planar surface comprises 384 or fewer microwells.

17. A method of analyzing a sample comprising:
(a) adding the sample to a microwell of one of claims 1-12 or one or more microwells of a microplate of one of claims 13-16; and
(b) using a detection instrument to detect a physical or chemical characteristic of the sample in the microwell or microwells.

18. The method of claim 17, wherein the instrument measures the physical or chemical characteristic of the sample from above or below the microwell or microwells.

19. The method of claim 17, wherein the instrument measures the physical or chemical characteristic at the center of the microwell.

20. The method of claim 19, wherein the instrument does not measure the physical or chemical characteristic at the periphery of the microwell.

21. The method of claim 17, wherein the instrument is a spectrophotometer and the physical or chemical characteristic is optical density.

22. The method of claim 17, further comprising a step of allowing the sample to incubate within the microwell or microwells.

23. The method of claim 17, further comprising repeating step (b) at one or more timepoints.

24. The method of claim 17, wherein the sample comprises one or more components capable of aggregation.

25. The method of claim 24, wherein the sample comprises one or more components capable of autoaggregation.

26. The method of claim 24, wherein the sample comprises one or more components capable of coaggregation.

27. The method of claim 24, wherein aggregates of the one or more components fall away from the center of the microwell or microwells along convex interior bottom surface toward the periphery of the microwell or microwells.

28. The method of claim 27, further comprising calculating the kinetics of aggregation based on the change in the physical or chemical characteristic measured at the center of the microwell or microwells over time.

29. The method of claim 28, comprising calculating the kinetics of coaggregation.

30. The method of claim 27, further comprising calculating the rate of aggregation based on the change in the physical or chemical characteristic measured at the center of the microwell or microwells over time.

31. The method of claim 30, comprising calculating the rate of coaggregation.

32. The method of claim 17, wherein the sample comprises one or more types of microbes.

33. The method of claim 32, wherein the sample comprises one or more species of bacteria.
